(19) United States
(12) Patent Application Publication
(10) Pub. No.: US 2011/0009718 A1
(43) Pub. Date: Jan. 13, 2011

(54) DETERMINATION OF PHYSIOLOGICAL PARAMETERS USING REPEATED BLOOD PRESSURE MEASUREMENTS

(76) Inventor: Benjamin GAVISH, Eshtaol (IL)

Correspondence Address: SUGHRUE MION, PLLC, 2100 PENNSYLVANIA AVENUE, N.W., SUITE 800, WASHINGTON, DC, 20037, US

(21) Appl. No.: 12/836,356

(22) Filed: Jul. 14, 2010

Related U.S. Application Data
(63) Continuation-in-part of application No. PCT/IL2009/000061, filed on Jan. 15, 2009.
(60) Provisional application No. 61/021,141, filed on Jan. 15, 2008.

(51) Int. Cl. A61B 5/021; A61B 5/1455; A61B 8/00

(52) U.S. Cl. 600/310; 600/485; 600/437

(57) ABSTRACT

A pulsewave detection unit is provided, at least a portion of the pulsewave detection unit being configured to be coupled to a portion of a subject's body, the pulsewave detection unit generating a signal that is responsive to arterial pressure of the portion of the subject's body. A pulsewave parameters determination unit receives respective first and second signals from the pulsewave detection unit, the signals being responsive to arterial pressure of the portion of the subject's body while the portion of the pulsewave detection unit that is coupled to the portion of the subject's body is at respective first and second heights, at respective times. An arterial parameters calculating unit determines an arterial property of the subject by processing the first and second signals, and generates an output in response to determining the arterial property. Other embodiments are also described.

### CROSS-REFERENCES TO RELATED APPLICATIONS

The present application is a continuation-in-part of PCT Application PCT/IL09/00061 to Gavish, filed Jan. 15, 2009, entitled “Determination of physiological parameters using repeated blood pressure measurements,” which claims the benefit of U.S. Provisional Patent Application 61/021,141 to Gavish, filed Jan. 15, 2008, entitled “Determination of physiological parameters using repeated blood pressure measurements.” All of the above applications are incorporated herein by reference.

### FIELD OF THE INVENTION

The present invention relates generally to medical devices. Specifically the present invention relates to external devices for evaluating arterial properties.

### BACKGROUND OF THE INVENTION

There is a growing interest in the relationship between the mechanical properties of arteries and cardiovascular diseases. The altered mechanical properties of arteries in common diseases, among them hypertension, diabetes and congestive heart failure, are typically related to the disease pathology, symptoms and risk of morbidity and mortality. Non-invasive methods of monitoring mechanical properties of arteries, mainly related to an artery's stiffness (or compliance, i.e., 1/stiffness), are becoming popular in clinical practice.

Blood pressure is a common physiological parameter used for diagnosis in both the clinic and the home setting. Blood pressure comprises two components, which are called respectively systolic blood pressure and diastolic blood pressure. Systolic blood pressure and diastolic blood pressure correspond respectively, to the maximum and minimum arterial pressure occurring during each cardiac cycle.

The difference between systolic pressure and diastolic pressure is called pulse pressure. The increase in arterial pressure during the cardiac cycle from diastole to systole is accompanied by a parallel increase in arterial volume. The difference between the maximum and the minimum arterial volume over the course of a cardiac cycle is called the pulse volume. The pulse volume per unit length of an artery is the pulse area of that artery.

Arterial stiffness G(P) may be defined by:

G(P)=dP(t)/dV(t) (Eq. 1)

where P(t) is the instantaneous pressure within the artery at time t, and V(t) is the volume of the artery at time t. Both P(t) and V(t) vary with time. A “linearized” definition of arterial stiffness, which is known in the art, is given by pulse pressure (PP) divided by pulse volume (ΔV). The linearized definition, and the definition provided by Eq. 1, provide the same results only in the case of a linear relationship between P(t) and V(t), in which G(P) is a constant. However, arteries typically become stiffer for greater arterial pressure, and therefore there is typically a nonlinear relationship between arterial pressure and arterial volume. (Throughout this application, the expressions “arterial stiffness” and “non-linearized arterial stiffness” refer to arterial stiffness as defined by Eq. 1. Linearized arterial stiffness is referred to as “linearized arterial stiffness.”)

Arterial stiffness may be similarly defined with respect to arterial cross-sectional area (Area) or arterial diameter (Diam), as opposed to arterial volume, i.e.:

G(P)=dP(t)/dArea(t), and

G(P)=dP(t)/dDiam(t).

(It is noted that there are several definitions of arterial stiffness that are used in the art, as summarized in an article by Gosling et al., entitled “Terminology for describing the elastic behavior,” Hypertension. 2003; 41: 1180-1182, which is incorporated herein by reference. Typically, arterial stiffness as used in the present application, is arterial stiffness as defined in Eq. 1. However, the scope of the present application includes using alternative definitions of arterial stiffness, mutatis mutandis, to obtain the results, relationships, and embodiments described in the present application, as is apparent to one skilled in the art.)

It is noted that pulse volume (ΔV) divided by pulse pressure (PP) is called “arterial capacitance”, as this ratio measures the ability of an artery to temporarily store blood in a way that smoothens the blood flow.

The following phenomenological relationships provide pressure-independent parameters (i.e., parameters that remain constant as arterial pressure is varied, unlike arterial stiffness, which varies as arterial pressure varies) that characterize the nonlinearity of the mechanical properties of arteries:

A) The Relationship Between Systolic Blood Pressure and Diastolic Blood Pressure:

A plot of systolic blood pressure (S) against diastolic blood pressure (D) in a subject whose blood pressure is monitored over an extended period of time (for example, 24 hours) commonly shows the following relationship:

S=A+ASI*D (Eq. 2)

where ASI is a subject-specific constant, called the Arterial Stiffening Index, by the inventor. ASI is the slope of the best-fit line of a plot of S versus D, the relationship between S and D having been assessed by Gavish et al., in an article entitled, “The linear relationship between systolic and diastolic blood pressure monitored over 24 hours: assessment and correlates,” J Hypertension 2008 26:199-209 (“Gavish 2008”), which is incorporated herein by reference. A related parameter is “Ambulatory arterial stiffness index” (“AASI”), as defined by Li et al. (2006) in an article entitled, “Ambulatory arterial stiffness index derived from 24-hour ambulatory blood pressure monitoring,” Hypertension 2006; 47:359-364. AASI is defined as:

AASI=1−(slope of best-line-fit of a plot of D versus S)

AASI was shown to be a predictor of cardiovascular mortality, in an article by Dolan et al., entitled “Ambulatory arterial stiffness index as a predictor of cardiovascular mortality in the Dublin Outcome Study,” Hypertension 2006; 47:365-370, which is incorporated herein by reference. Similarly, in (a) a reference entitled “A modified ambulatory arterial stiffness index is independently associated with all-cause mortality,” Journal of Human Hypertension (2008) 22, 761-766, and (b) a reference entitled “Measures of the Linear Relationship Between Systolic and Diastolic Ambulatory Blood Pressure Predict All-Cause Mortality,” Ben-Dov et al., Abstract presented at the 61st Annual High Blood Pressure Research Conference 2007, Tucson, Ariz., Sep. 26-29, 2007, the authors describe the relationship between S and D as being related to mortality.

(It is noted that ASI and AASI are mathematically related, as demonstrated in the “Gavish 2008” article. Therefore, the scope of the present invention includes using AASI instead of ASI, mutatis mutandis, to obtain the results, relationships, and embodiments described in the present application, as is apparent to one skilled in the art.)

More specifically, ASI has been shown to measure the tendency of arteries to become stiffer during systole in an article by Gavish, entitled “Repeated blood pressure measurements may probe directly an arterial property,” Am. J. Hypertension 2000; 13:19 A (“Gavish 2000”), which is incorporated herein by reference.

According to Gavish 2000,

ASI=G(S)/G(D) (Eq. 3)

Gavish 2008 describes the fact that ASI is frequently in the range of 1 to 2.

### B) The Pressure Dependence of Arterial Stiffness:

Differential arterial stiffness (G(P)) has been shown to increase linearly with pressure in an article by Pagani, entitled “Effects of age on aortic pressure-diameter and elastic stiffness-stress relationships in unanesthetized sheep,” Circ. Res. 1979; 44; 420-429, and in an article by Gavish, entitled “The pressure dependence of arterial compliance: A model interpretation,” Am J Hypertens 2001; 14:121 A (“Gavish 2001”), which is incorporated herein by reference.

The slope of a curve which plots differential arterial stiffness against pressure (dG(P)/dP) is a pressure-independent physiological parameter that characterizes the tendency of arteries to stiffen with elevating pressure. The slope of this curve has been shown to have a different range of values for subjects suffering from cardiovascular diseases, compared to that of healthy subjects in Gavish 2001.

C) The Height Dependence of Systolic Blood Pressure and Diastolic Blood Pressure:

The dependence of blood pressure on the height of the measurement site, with respect to an arbitrary reference height, is a known phenomenon. For example, see “Textbook of Medical Physiology” (Guyton A C and Hall J E, W.B.Saunders, Philadelphia, 9th edition, Chapter 14, pp. 161-181), which describes this phenomenon as reflecting a hydrostatic effect. For example, blood pressure at a site that is at a lower level than the heart will be greater than blood pressure at the heart, due to the additional pressure exerted by a column of blood between the heart and the measuring site. According to this effect, blood pressure increases by approximately 8 mm Hg for 10 cm lowering of the measuring site. Therefore, in accordance with Guyton, one would expect that plotting systolic pressure and diastolic pressure against height would result in two parallel lines having the same slope.

The following patents may be of interest:

U.S. Pat. No. 4,779,626 to Peel

U.S. Pat. No. 4,998,534 to Claxton

U.S. Pat. No. 5,103,833 to Apple

U.S. Pat. No. 5,201,319 to Negishi

U.S. Pat. No. 5,778,879 to Ota et al

U.S. Pat. No. 5,778,979 to Burleson

U.S. Pat. No. 6,309,359 to Whitt

U.S. Pat. No. 6,662,032 to Gavish

U.S. Pat. No. 6,872,182 to Kato

U.S. Pat. No. 7,101,338 to Yang

WO 09/090,646 to Gavish

The following references, which may be of interest, are incorporated herein by reference:

“Biometry,” Sokal R R and Rohlf F J (1981), 2nd ed. Chap 15 pp. 561-616, Freeman, New York

“Plethysmographic characterization of vascular wall by a new parameter—minimum rise time: Age dependence in health,” Gavish B., Microcirc Endothel lymph. 1987: 3; 281-296,

“Practical Noninvasive Vascular Diagnosis,” Kempczinski RF and Yao JST (1982), Year Book Medical Publishers, Chicago

“Pulse wave analysis,” O'Rourke M F et al., Br J Clin Pharmacol, 2001; 51; 507-522

“Regression Analysis for Social Science,” von Eye A, and Schuster C, Academic Press, San Diego, 1998, Chap 12, pp. 209-236

“The nonlinearity of pressure-diameter relationship in arteries as a source for pulse pressure widening: A model view,” Gavish, Abstract #1547 presented in the meeting of the European Society of Hypertension, Milan, Jun. 15-17, 2006 (“Gavish 2006”)

“Velocity of transmission of the pulse and elasticity of arteries,” Bramwell J. C., and Hill A. V., Lancet 11922, 891-892

Micro Medical Ltd (Kent, UK) manufactures the PulseTrace PWV, which is described as measuring arterial stiffness between two locations of the arterial tree.

### SUMMARY OF THE INVENTION

It has been discovered by the inventor that systolic blood pressure and diastolic blood pressure show different dependencies on height, as shown in FIG. 1C.

The following relationships have been shown by the inventor to exist between systolic blood pressure (S(H)) and height (H), and between diastolic blood pressure (D(H)) and height (H):

S(H)=As−Bs*H

where Bs=dS(H)/dH (Eq. 4)

D(H)=Ad−Bd*H

where Bd=dD(H)/dH≠Bs (Eq. 5)

where As, Bs, Ad, and Bd are generally constant for a specific subject, and where Bd and Bs are not necessarily equal. In some embodiments of the invention, Bs and Bd of a subject are determined by regression analysis, as described hereinbelow.

Eqs. 4 and 5 can be combined with Eq. 2 to give the following relationship between the arterial stiffening index (ASI), the derivative of systolic blood pressure with respect to height (Bs), and the derivative of diastolic blood pressure with respect to height (Bd):

ASI=Bs/Bd (Eq. 6)

It follows from the fact that ASI is typically not equal to 1 that the derivative of systolic pressure with respect to height is typically different from that the derivative of diastolic pressure with respect to height, as shown in FIG. 1C.

All three phenomena can be linked to arterial properties using a well-known phenomenological pressure-volume relation that holds in arteries:

P=u+v*exp(V/Ve) (Eq. 7)

where, u, v and Ve are pressure-independent adjustable constants, where Ve is called here “arterial expansivity,” and where the parameter u is the “zero-stiffness pressure.”

The inverse of arterial expansivity (i.e., 1/Ve) is known as “stiffness constant” (as explained in an article by Stefanadis et al., entitled “Pressure-diameter relation of the human aorta,” Circulation 1995; 92:2210-2219).

The zero stiffness pressure u is a pressure lower than which the present model is less valid, due to the phenomenon of arterial collapse that occurs when the pressure outside of an artery is sufficiently greater than the arterial pressure. The parameter u can be calculated from the parameters A and ASI given by Eq. 2 as follows:

u=−A(ASI−1) (Eq. 7a)

It follows that the following relationship exists between arterial stiffness (G(P)) and arterial pressure (P):

G(P)=dP/dV=(P−u)Ne (Eq. 8)

Given Eq. 8, the slope of G(D) plotted versus D, or of G(S) plotted versus S (and generally G(P) plotted versus P) can provide the stiffness constant, 1/Ve, i.e.:

dG/dP=1/Ve (Eq. 9)

Using techniques described in Gavish 2000 and Gavish 2001, both of which articles are cited hereinabove, one sees that Eq. 8 leads to the following expression, relating the arterial stiffening index (ASI) to systolic arterial stiffness (G(S)), diastolic arterial stiffness (G(D)), and the pulse volume (ΔV):

ASI=G(S)/G(D)=exp(ΔV/Ve) (Eq. 10)

Eq. 10 shows that if ΔV is measured, in addition to blood pressure measurements from which ASI is determined, then Ve may be calculated by

Ve=ΔV/ln(ASI) (Eq. 11)

According to Eq. 10, ASI=1 corresponds to an elastic artery with pressure-independent stiffness and a linear pressure-volume relationship. The amount by which ASI exceeds 1 corresponds to the non-elastic nature of an artery, and is associated with its tendency to stiffen upon elevating arterial pressure, thereby reflecting the deviation of the artery pressure-volume relationship from linearity. The reference by Gavish, entitled, “The nonlinearity of pressure-diameter relationship in arteries as a source for pulse pressure widening: A model view,” Abstract #1547 presented in the meeting of the European Society of Hypertension, Milan, Jun. 15-17, 2006 (“Gavish 2006”), which is incorporated herein by reference, shows that by using the arterial stiffening index of an artery, as calculated by Eq. 2 using blood pressure measurements, with its interpretation following Eq. 10, one can split the pulse pressure into components associated with the elastic and non-elastic nature of an artery. Typically, the inventor hypothesizes, the components of the pulse pressure which are associated with a non-linear pressure-volume relationship are associated with cardiovascular risk factors. Therefore, being able to determine these components in clinical practice, as provided by some embodiments of the present invention, is a useful diagnostic tool or tool that can be used along with others to aid a physician in making a diagnosis.

(It is noted that all of the results and relationships demonstrated herein that involve arterial volume apply equally for arterial cross-sectional area and arterial diameter, mutatis mutandis. Accordingly, the results and relationships described with respect to the “pulse volume” hold equally for “pulse area” and “pulse diameter.” Therefore the scope of the present invention includes using pulse area and/or pulse diameter instead of pulse volume, mutatis mutandis, as would be apparent to one skilled in the art.)

Gavish 2006 derives from Eq. 10 a relationship between the components of the pulse pressure (PP) that have a linear relationship with arterial volume (PP-elastic), the components of the pulse pressure that have a non-linear relationship with arterial volume (PP-nonelastic), and the arterial stiffening index (ASI).

By definition, PP-elastic is determined by:

PP-elastic=G(D)·ΔV (Eq. 12)

Furthermore, PP-nonelastic is PP minus PP-elastic. Therefore, using Eq. 12 and the relationships demonstrated hereinabove, the inventor has demonstrated in Gavish 2006:

PP-nonelastic/PP-elastic=((ASI−1)/ln(ASI))−1 (Eq. 13)

PP-elastic=PP−(ln(ASI))/(ASI−1) (Eq. 13a)

PP-nonelastic=PP−[1−(ln(ASI))/(ASI−1)] (Eq. 13b)

For example, based on Eq. 13, if ASI=1.1, PP-nonelastic/PP-elastic=0.05, suggesting that the nonelastic component of the pulse pressure is only 5% of the elastic component. On the other hand, if ASI=2 then the corresponding ratio is 44%.

The inventor has derived a relationship between diastolic arterial stiffness in the form given by Eq. 1, i.e., G(D)=dD/dV, and the above-mentioned linearized form (PP/ΔV), by combining Eq. 12 and Eq. 13 into

G(D)=(PP/ΔV)−(ln(ASI))/(ASI−1) (Eq. 14)

The systolic arterial stiffness G(S) can then be determined from diastolic arterial stiffness G(D), using Eq. 10.

Furthermore, since

ASI=G(S)/G(D), and

ASI=Bs/Bd,

the inventor hypothesizes that the derivative (Bs) of systolic blood pressure with respect to height is proportional to systolic arterial stiffness (G(S)) at the mean value of systolic pressure (S), and the derivative of diastolic blood pressure with respect to height (Bd) is proportional to diastolic arterial stiffness (G(D)), at the mean value of diastolic pressure (D).

It is noted that although a mathematical basis for the inventor's hypotheses has been set out herein, the scope of the present invention is not limited to embodiments which correspond to the hypotheses.

It is noted that in some embodiments of the invention, the term “blood pressure measurement” includes an outcome of processing a blood pressure signal generated by a blood pressure sensor at a measuring site.

In some embodiments of the present invention, the blood pressure of a subject (or another measurement that is responsive to arterial pressure) is measured while a portion of the subject's body, to which a measuring device is coupled, is at a first height with respect to a reference height. The portion of the subject's body is moved to a second height with respect to the reference height, and the subject's blood pressure (or the other measurement) is measured a second time when the portion of the subject's body is at the second height. A physiological parameter of the subject is determined by processing the blood pressure measurements (or the other measurement) and, optionally, an indication regarding the first and second heights, and an output is generated in response to determining the physiological parameter.

Repeated blood pressure (BP) measurements taken over a relatively wide range of heights are typically taken in order to determine many of the parameters that characterize an artery, which are described hereinabove. Changing the height of a limb with respect to the heart level may be a systematic way of changing BP locally without affecting BP in the whole body. Therefore, in some embodiments, the height of a limb is varied systematically in order to vary blood pressure systematically.

In some embodiments of the invention, a set of one or more arterial properties are derived by repeatedly measuring blood pressure while placing the blood pressure measuring site at different heights with respect to a reference point. Alternatively or additionally, the pulse volume, the pulse diameter, the pulse area, pulsewave pattern geometrical characteristics, and/or pulsewave velocity are measured and/or derived. These parameters, all or some of which are measured and/or derived at a number of different heights, in accordance with embodiments of the invention, are collectively described as “pulsewave characteristics” or “pulsewave parameters” in this application (since all of these measurements are associated with a waveform that pulsates).

Pulsewave geometrical characteristics may include, for example, a rate of change of pulse pressure, pulse rise time, pulse decay time, duration between time points corresponding to systole and/or diastole, and/or a relative amplitude of the pulsewave.

The pulsewave characteristics are typically determined using techniques that are known in the art, as described, for example in the following references, which are incorporated herein by reference:
Pagani (1979), cited hereinabove,
“Practical Noninvasive Vascular Diagnosis,” Kempczinski RF and Yao JST (1982), Year Book Medical Publishers, Chicago, which describes:
ultrasonic determination of arterial diameter in Part II, chapter 2, entitled “Ultrasound,” by Summer D S, pp 21-47,
pulse volume and wave form in Part III, chapter 7 entitled “Segmental volume plethysmography: the pulse volume recorder,” by Kempczinski R F, and in Part III, chapter 3, entitled “Plethysmography,” by Yao J T and Flinn W R,
“Velocity of transmission of the pulse and elasticity of arteries,” Bramwell J. C., and Hill A. V., Lancet 11922, 891-892, which describes determination of arterial stiffness by pulsewave velocity,
“Pulse wave analysis,” O'Rourke M F et al., J Clin Pharmacol, 2001; 51; 507-522, which describes the analysis of a pulse waveform.

These techniques are applied commercially, for example, by Micro Medical Ltd in the PulseTrace PWV, described hereinabove.

The pulsewave characteristics are measured by one or more sensors disposed at the blood pressure measurement site. In some embodiments, the sensor includes a cuff, an intravascular pressure sensor, a photoplethysmogram (PPG), and/or a strain gauge plethysmograph. In some embodiments, the sensor includes a cuff that applies a force on the circumference of a body portion at the measuring site. In some embodiments, the sensor detects blood properties that change with pressure, e.g., spectral properties of hemoglobin. For example, a finger-mounted PPG may be placed on a subject's finger and measure blood pressure in the subject's finger while the subject moves his/her hand up and down.

As described hereinabove, a physiological parameter of the subject is determined by processing the blood pressure measurements (or another pulsewave parameter) and, optionally, an indication regarding the first and second heights, and an output is generated in response to determining the physiological parameter. Typically, the physiological parameter is an arterial property of the subject, e.g., a mechanical property of the subject's arteries. For example, one or more of the following arterial properties may be determined:

arterial stiffening index;

ambulatory arterial stiffness index;

the change of stiffness with change of pressure (dG(P)/dP);

the derivative of systolic blood pressure with respect to height (dS(H)/dH);

the derivative of diastolic blood pressure with respect to height (dD(H)/dH);

arterial expansivity

stiffness constant; and

zero-stiffness pressure.

(In general, in the context of the present application, the term “pulsewave parameter” is used to describe parameters that are determined from, or derived based upon, one or more measurements of one or more parameters of the subject's pulsewave at a single height. For example, pulsewave parameters may include blood pressure, pulse area, pulse diameter, pulse volume, and/or arterial capacitance. In the context of the present application, parameters that are determined in response to the determination of a plurality of pulsewave parameters at respective heights are called “arterial properties.”)

In some embodiments of the invention, ASI is determined by taking repeated blood pressure measurements at different heights, as described hereinbelow, and using Eq. 2. The height of the blood pressure measurement site is varied in order to provide a range of values for S and D, from which the ASI can be determined.

In some embodiments, the ratio that relates the elastic components of the pulse pressure to the nonelastic components thereof are determined from the ASI, using Eq. 12, and/or the absolute value of these components is determined from ASI and the pulse pressure, using Eq. 13a.

In some embodiments, at each of the heights at which a blood pressure measurement is measured, pulse volume, pulse area, and/or another parameter related to pulse volume is measured. Using Eq. 14, the systolic and/or the diastolic value of the arterial stiffness are determined. In some embodiments, arterial expansivity is determined using Eq. 9 or 11. In some embodiments, having calculated the value of systolic or diastolic arterial stiffness from Eq. 14, and the value of the arterial expansivity using Eq. 9 or 11, the zero-stiffness pressure is calculated using Eq. 8.

For some applications, for each of the heights at which a blood pressure measurement is taken, the height of the blood pressure measuring site with respect to a reference height is measured or estimated, and the derivative of systolic blood pressure with respect to height (Bs), and/or the derivative of diastolic blood pressure with respect to height (Bd) is determined, using Eqs. 4 and 5. In some embodiments, the ASI is calculated or verified using the values determined for Bs and Bd, and Eq. 6.

In some embodiments, the height of the blood pressure measuring site is measured or estimated using techniques that are known in the art, for example, by manually measuring the height and keying in the height on a user interface. Alternatively or additionally, data associated with the position of a support structure that supports the blood pressure measuring site during the measuring is keyed in to a user interface, or is detected by sensors. For example, the position of the support structure may be detected by detecting the hydrostatic pressure generated by a fluid-filled tube that is coupled to the support structure, using techniques described in U.S. Pat. No. 4,779,626, which is cited hereinabove, and which is incorporated herein by reference. Alternatively, or additionally, the height of the blood pressure measuring site is determined using a 3D acceleration chip that detects the spatial position of a blood pressure sensor, the blood pressure measuring site, and/or a support structure as described hereinabove, using techniques described in U.S. Pat. No. 7,101,338, which is cited hereinabove, and which is incorporated herein by reference.

It is noted that although some of the embodiments described herein describe the use of blood pressure measurements or blood pressure signals for determining arterial parameters of a subject, the scope of the present invention includes using other measurements for determining arterial parameters of the subject. For example, pulse volume, pulse area, pulse diameter, a flow rate, spectral characteristics, and/or a different parameter of the subject's blood may be measured, mutatis mutandis, for determining the subject's arterial parameters.

The present invention will be more fully understood from the following detailed description of embodiments thereof, taken together with the drawings, in which:

### BRIEF DESCRIPTION OF THE DRAWINGS

FIG. 1A is a schematic illustration of an arm cuff being positioned at different heights, in accordance with an embodiment of the present invention;

FIG. 1B is a graph showing the relationship between systolic and diastolic blood pressure, the blood pressures having been measured using the arm cuff of FIG. 1A, in accordance with an embodiment of the present invention;

FIG. 1C is a graph showing the relationship between systolic and diastolic blood pressure and the height of the blood pressure measuring site, the blood pressures having been measured using the arm cuff of FIG. 1A in accordance with an embodiment of the present invention;

FIG. 2A is a schematic illustration of a wrist cuff being positioned at different heights, in accordance with an embodiment of the present invention;

FIG. 2B is a graph showing the relationship between systolic and diastolic blood pressure, the blood pressures having been measured using the wrist cuff of FIG. 2A in accordance with an embodiment of the present invention;

FIG. 2C is a graph showing the relationship between systolic and diastolic blood pressure and the height of the blood pressure measuring site, the blood pressures having been measured using the wrist cuff of FIG. 2A, in accordance with an embodiment of the present invention;

FIGS. 3A-B are block diagrams of blood pressure measurement apparatus, in accordance with respective embodiment of the present invention;

FIG. 4 is a flowchart showing the operation of the blood pressure measurement apparatus, in accordance with an embodiment of the present invention;

FIG. 5 is a flowchart showing the process of determining physiological parameters of a subject, in accordance with an embodiment of the present invention;

FIG. 6 is a schematic illustration of an operational input unit for use with a cuff that measures blood pressure, in accordance with an embodiment of the present invention;

FIG. 7 is a schematic illustration of an operational input unit for use with a cuff that measures blood pressure and pulse volume, in accordance with an embodiment of the present invention;

FIG. 8 is a schematic illustration of apparatus for measuring blood pressure and for manually receiving information regarding the height of the blood pressure measuring site, in accordance with an embodiment of the present invention;

FIG. 9 is a schematic illustration of apparatus for measuring blood pressure and pulse volume and for manually receiving information regarding the height of the blood pressure measuring site, in accordance with an embodiment of the present invention;

FIG. 10 is a schematic illustration of apparatus for measuring blood pressure and for receiving information regarding the height of the blood pressure measuring site via a sensor, in accordance with an embodiment of the present invention;

FIG. 11 is a schematic illustration of apparatus for measuring blood pressure and pulse volume and for receiving information regarding the height of the blood pressure measuring site via a sensor, in accordance with an embodiment of the present invention; and

FIG. 12 is a schematic illustration of a support structure for supporting a blood pressure measuring site, in accordance with an embodiment of the present invention.

### DETAILED DESCRIPTION OF THE INVENTION

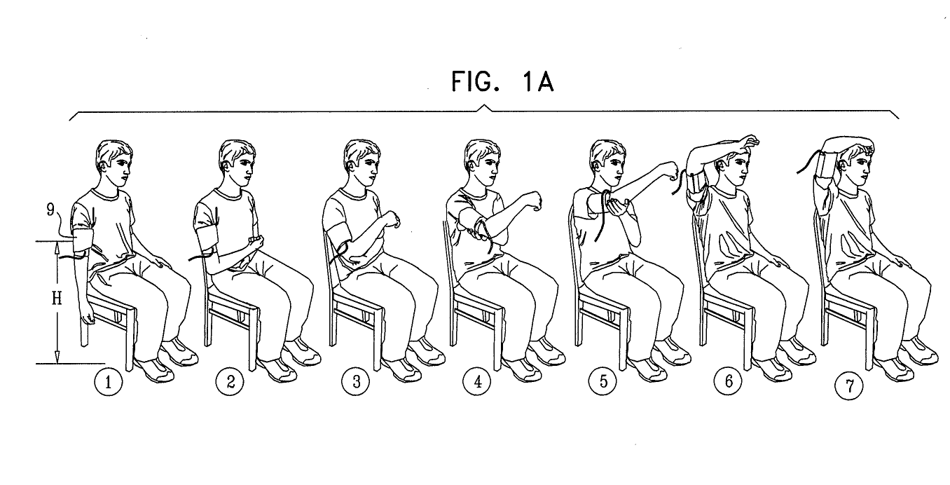

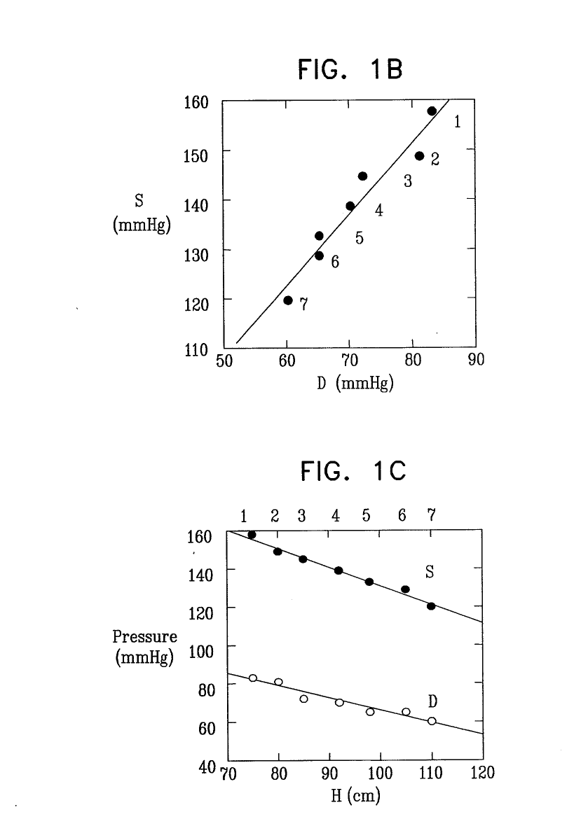

Reference is now made to FIG. 1A which is a schematic illustration of a cuff 9 being positioned at different heights H (“the cuff height”), in accordance with an embodiment of the present invention. The cuff height is measured between an arbitrary reference height, e.g., the floor, to a position on the cuff such as the center of the cuff, as shown. In some embodiments (as shown in FIG. 1A), the cuff is placed around the subject's arm (i.e., an “arm cuff”), and the subject assumes a number of different postures (i.e., body positions), in order to position the cuff at a number of different heights.

In some embodiments, a number of cuff heights, having almost constant cuff-height intervals between successive cuff heights are determined as follows. The maximum and minimum cuff heights that allow a user to position him/herself comfortably are determined. The difference between the maximum and minimum heights is divided into (n−1) intervals. The subject first assumes a position at which the cuff height is at the minimum, and takes measurements as described herein. Subsequently, the subject raises the cuff height by one height interval, and repeats the measurements. The subject continues to raise the height of the cuff by incremental intervals and taking measurements, until the cuff height is at the maximum cuff height. Typically, the subject holds the cuff at each of the cuff heights by supporting his/her arm with the other hand, with a different part of the body, or with an accessory, e.g., a table, in order to stabilize the subject's posture without causing discomfort to the subject. Typically, the arm on which the cuff is placed is supported at a position other than the position on the arm on which the cuff is placed in order to prevent deformation of the cuff. This procedure for determining cuff heights may be applied to all types of cuffs mentioned in this application, mutatis mutandis.

While the cuff is at each of the heights, a blood pressure measurement, and/or other measurements are measured by the cuff. For example, as shown, the subject may assume seven different postures. In posture 1 the hand hangs freely, and the cuff height is at a minimum. In posture 2, the hand is placed on the abdomen, and in posture 3, in which the cuff is positioned at heart level, the hand is slightly raised and supported by the other hand. Posture 4 is similar to posture 3, but the arm is positioned at shoulder level, parallel to the floor. Posture 5 is similar to posture 4, but the arm is slightly raised above shoulder level, such that the cuff is level with the subject's neck. In posture 6, the back of the hand is placed on the forehead, such that the cuff is level with the subject's mouth, and in posture 7 the forearm is fully supported by the head, the cuff being level with the subject's ear. Typically, postures are chosen such that by the user assuming a given posture, the arm on which a measurement is taken is supported and the cuff in unconstrained. In some embodiments, the arm is positioned in a set of postures in which the angle between the arm and the forearm is nearly constant.

Reference is now made to FIG. 1B, which is a graph showing the relationship between systolic (S) and diastolic (D) blood pressure, the blood pressures having been measured using arm cuff 9 of FIG. 1A, in accordance with an embodiment of the present invention. The data were measured with a standard digital blood pressure monitor, the arm cuff having been positioned by the subject assuming the postures shown in FIG. 1A. In the plotted example, the correlation coefficient r between S and D was found to be 0.969, and the estimated value of the slope of the line, i.e., the ASI, was 1.500±0.144 (mean±standard error of mean, using a symmetric type of regression, as described in Gavish 2008).

Reference is now made to FIG. 1C, which is a graph showing the relationship between systolic (S) and diastolic (D) blood pressure and the height of the blood pressure measuring site, the blood pressures having been measured using arm cuff 9 of FIG. 1A, in accordance with an embodiment of the present invention. The correlation coefficients of systolic and diastolic pressure with the height of the measuring site were found to be 0.992 and 0.973 respectively. The derivative of systolic blood pressure with respect to height (Bs) was found to be −0.941±0.048 mmHg/cm, and the derivative of diastolic blood pressure with respect to height (Bd) was found to be −0.662±0.059 mmHg/cm. Bs divided by Bd was thus 1.497±0.076, which is similar to the above estimation for ASI.

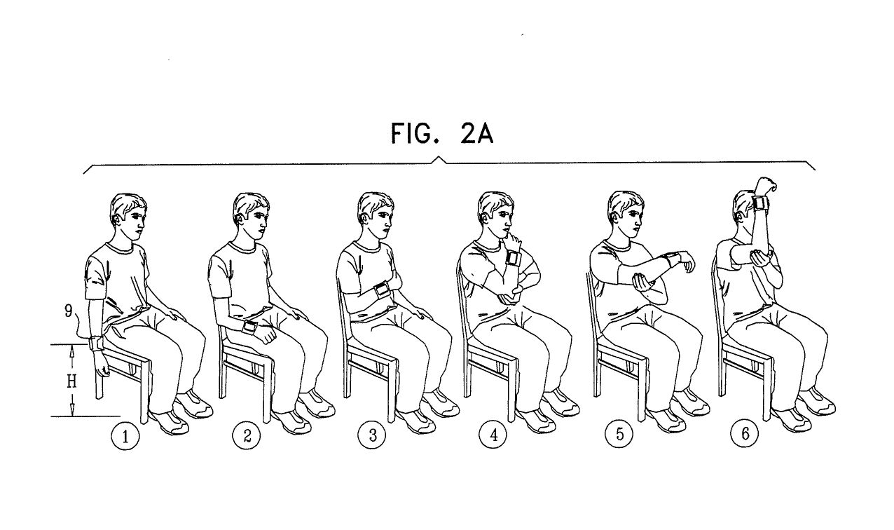

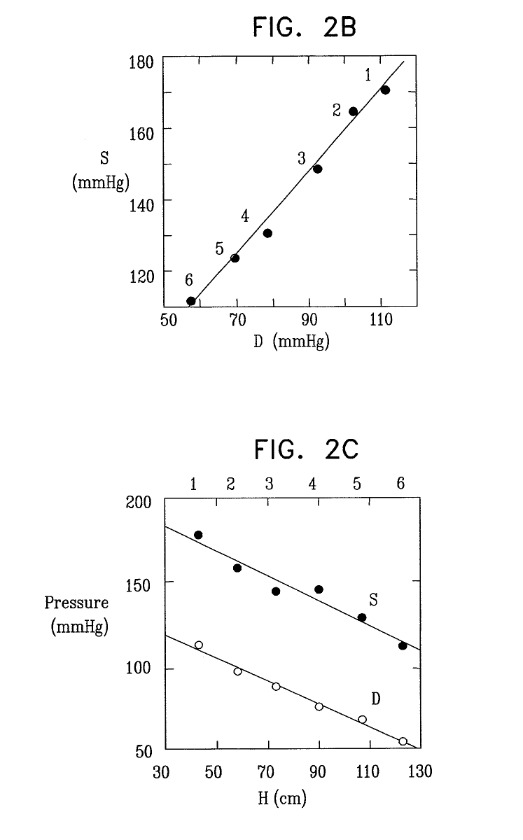

Reference is now made to FIG. 2A which is a schematic illustration of cuff 9 being positioned at different heights H, in accordance with an embodiment of the present invention. In some embodiments (as shown in FIG. 2A), the cuff (a “wrist cuff”) is placed around the subject's wrist, and the subject assumes a number of different postures, in order to position the cuff at a number of different heights H. While the cuff is at each of the heights, a blood pressure measurement, and/or other measurements are measured by the cuff. For example, as shown, the subject may assume six different postures. In posture 1, the hand hangs freely, the cuff height being at a minimum. In posture 2, the hand is placed on the side of the thigh, and in posture 3 the wrist is placed horizontally on the abdomen. In posture 4, in which the cuff is positioned at heart level, the elbow is supported by the other hand. In posture 5, the forearm is positioned horizontally at the height of the shoulders. In posture 6, the forearm is positioned vertically, such that the cuff is level with the subject's forehead. In some embodiments, the arm is positioned in a set of postures in which the angle between the forearm and the palm of the hand is nearly constant.

Reference is now made to FIG. 2B which is a graph showing the relationship between systolic (S) and diastolic (D) blood pressure, the blood pressures having been measured using wrist cuff 9 of FIG. 2A, in accordance with an embodiment of the present invention. The data were measured with a standard digital blood pressure monitor, the wrist cuff having been positioned by the subject assuming the postures shown in FIG. 2A. In the plotted example, the correlation coefficient r between S and D was found to be 0.980, and the estimated value of the slope of the line, i.e., the ASI, was 1.044±0.105.

Reference is now made to FIG. 2C, which is a graph showing the relationship between systolic (S) and diastolic (D) blood pressure and the height of the blood pressure measuring site, the blood pressures having been measured using wrist cuff 9 of FIG. 2A, in accordance with an embodiment of the present invention. The correlation coefficients of systolic and diastolic pressure with the height of the measuring site were found to be 0.963 & 0.993 respectively. The derivative of systolic blood pressure with respect to height (Bs) was found to be −0.775±0.108 mmHg/cm, and the derivative of diastolic blood pressure with respect to height (Bd) was found to be −0.748±0.044 mmHg/cm. Bs divided by Bd was thus 1.036±0.117, which is similar to the above estimation for ASI.

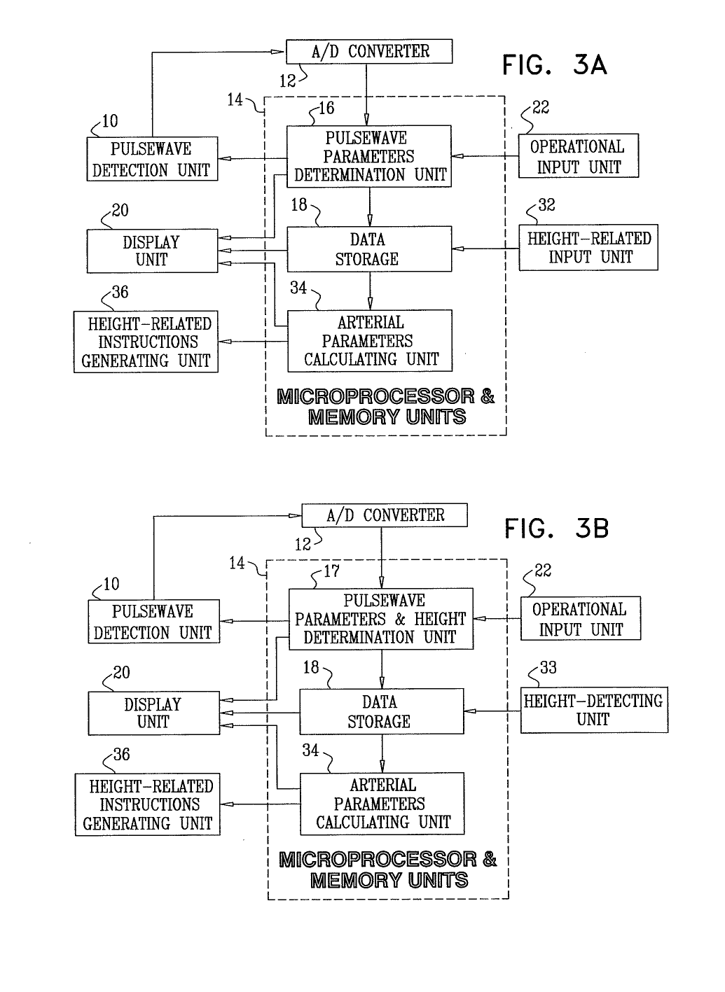

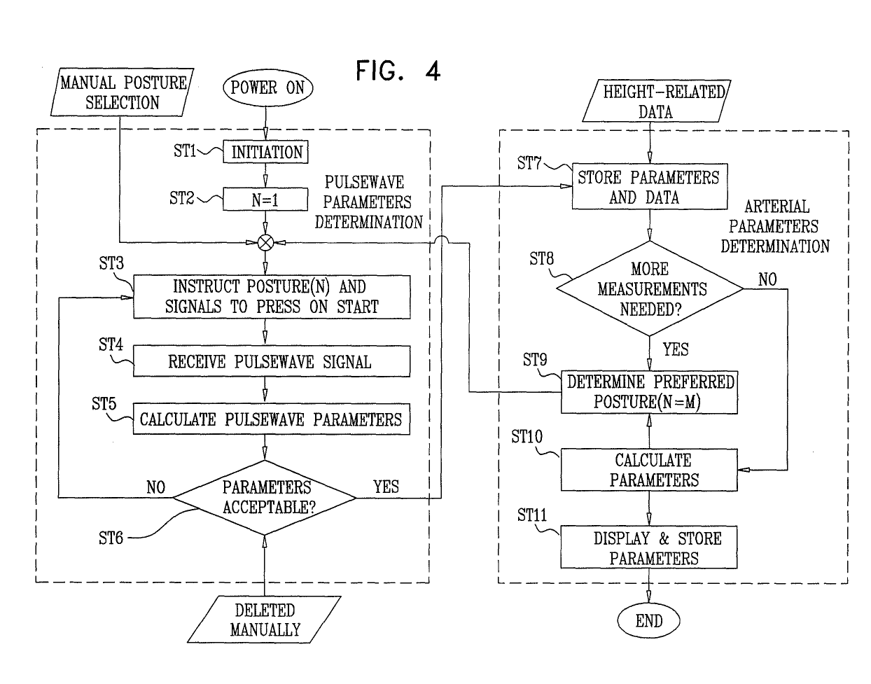

Reference is now made to FIGS. 3A-B, which are block diagrams of blood pressure measurement apparatus, in accordance with an embodiment of the present invention. A pulsewave detection unit 10 typically comprises a cuff (e.g., cuff 9) fastened to a user's arm (as shown in FIG. 1), wrist (as shown in FIG. 2), ankle, or finger, with air pressure controlled by pressurizing and exhausting units (not shown) that are controlled by a microprocessor or manually. The pulsewave detection unit includes a pressure sensor (not shown) that generates a signal. (In some embodiments, the signal-generating sensor is disposed remotely from the cuff, the cuff being coupled to a portion of the subject's body, as described herein. For example, the pressure detected by the cuff may be conveyed to a sensor that is disposed inside a control unit, the sensor generating an electrical signal in response to the detected pressure.) This part of the apparatus is currently used in standard commercial electronic home blood pressure monitors.

In some embodiments, pulsewave detection unit 10 includes a subunit that generates a signal from which the cuff volume can be calculated, using techniques described hereinabove, in the Background and in the Summary. For example, techniques may be used that are described in U.S. Pat. No. 5,103,833 to Apple et al., or in “A new oscillometry-based method for estimating the brachial arterial compliance under loaded conditions,” by Liu S H, Wang J J, Huang K S, IEEE Trans Biomed Eng. 2008 55:2463-2470, both of which references are incorporated herein by reference.

Alternatively or additionally, the pulsewave detection unit measures arterial diameter. Arterial diameter is typically measured using ultrasonic tracking. In some embodiments, the arterial cross section area is calculated, using the arterial diameter measurement. For some applications, the pulsewave detection unit measures pulsewave velocity, and/or pulsewave pattern geometrical characteristics in accordance with techniques described in references cited hereinabove, in the Summary (for example, the following references cited hereinabove, which are incorporated herein by reference: Kempczinski et al (1982), Bramwell et al. (1922), O'Rourke et al. (2001), Gavish (1987)).

In some embodiments, the operation of pulsewave detection unit 10 is controlled by operational input unit 22, via a pulsewave parameter determination unit 16. The control of the pulsewave detection unit may include, for example, starting and stopping a measurement, selecting to perform a single measurement as a simple BP determination, or a series of measurements useful for calculating physiological parameters, and selecting from a menu in order to customize an operation, for example, by accessing stored data. For some applications, the status of the measurements and its control are provided to the user by a display unit 20. In some embodiments, signals generated by the pulsewave detection unit are digitized by an analog to digital converter 12 and processed by a microprocessor 14.

In some embodiments, the microprocessor includes a pulsewave parameter determination unit 16 that determines BP and pulse volume (if measured) and all other parameters that can be derived from the pulsewave that may be associated with pressure-dependent arterial properties. For example, the determination unit may determine a rise time of arterial pressure (for example, “minimum rise time” as defined by Gavish B., in an article entitled “Plethysmographic characterization of vascular wall by a new parameter—minimum rise time: Age dependence in health,” Microcirc Endothel Lymph. 1987: 3; 281-296, which is incorporated by reference) or a decay time of arterial pressure. In some embodiments, the data are stored in a data storage 18, and/or are displayed by the display unit. For some applications, data storage 18 also stores previous pulsewave measurements and physiological data that can be erased or downloaded following the input provided by the operational input unit 22.

In some embodiments, an arterial parameters calculating unit 34 analyzes parameters that can be derived from a series of data points, e.g., the slope of the line shown in FIG. 1B. By performing such a calculation, this unit also identifies deviations of specific data from a predicted behavior and can generate a message requesting the user to repeat a measurement, or identify a benefit for performing additional measurements. Arterial parameters calculating unit 34 also activates guiding of the user to position the pulsewave detection unit at different heights suitable for appropriate determination of the physiological parameters. The guiding is delivered to the user via display unit 20 or via a height-related instructions generating unit 36 that generates additional stimuli to the user, such as voice messages.

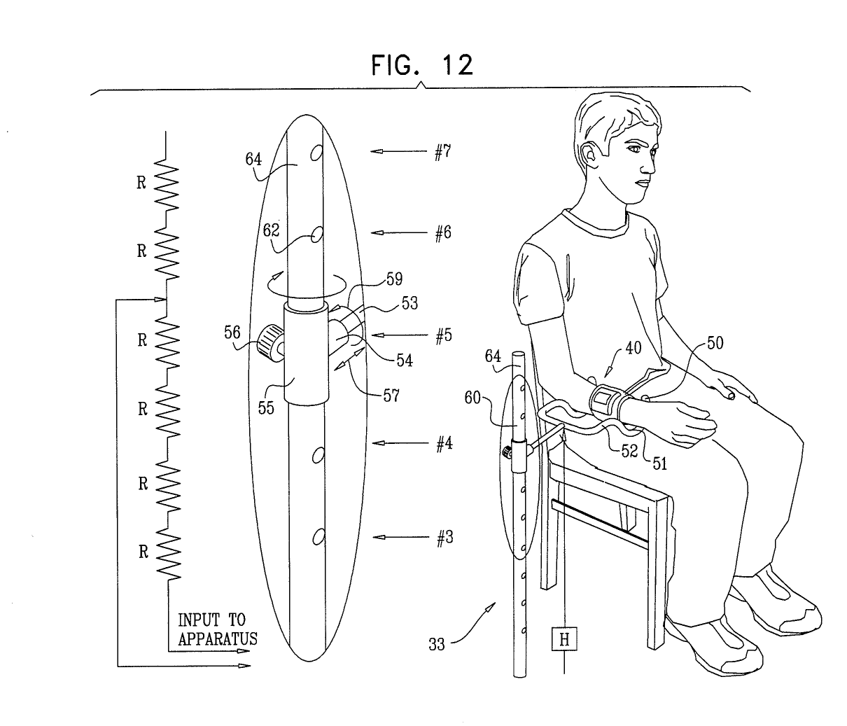

In some embodiments, display unit 20 or height-related instructions generating unit 36 guides the user to adopt assume a specific posture or to move the organ on which the cuff is mounted to a given spatial orientation. For example, in embodiments in which different heights of the measuring site are achieved by the user assuming different postures, as illustrated in FIG. 1A and FIG. 2A, the height-related command may illustrate a specific posture to generate. In some embodiments, a height indication (indicating the height of pulsewave detection unit 10, for example) is keyed in using a height-related input unit 32. This information can be the height measured directly from an arbitrary reference, e.g., a floor, by the user, using a meter stick. In some embodiments a support structure assists in positioning the blood pressure measuring site at a preferred posture and provides height information (indicating the height of pulsewave detection unit 10, for example) directly or indirectly via codes. Such a structure is described hereinbelow with reference to FIG. 12.

In some embodiments, arterial parameters calculating unit 34 detects deviant height-related measurements using the linear relationship between blood pressure and height.

The apparatus shown in FIG. 3B is generally similar to that of FIG. 3A. The apparatus of FIG. 3A includes height-related input unit 32 via which height-related data are manually entered. The apparatus of FIG. 3B includes a height detecting unit 33 that generates a signal, from which the height of (for example) the center of gravity of the body part of the user that generates the detected pulsewave signal, the cuff height, described with reference to FIGS. 1A and 2A, or the height of a different pulsewave detection unit 10, or a different portion of the pulsewave detection unit, is determined. Such signals are generated, for example, by sensing the hydrostatic pressure in a fluid-filled tube, as described in U.S. Pat. No. 4,779,626, which is incorporated herein by reference, by using a 3D acceleration chip that detects the spatial position, as described in U.S. Pat. No. 7,101,338, which is incorporated herein by reference, and/or via codes provided by a supporting structure, as described with reference to FIG. 12. Accordingly, the pulsewave parameters determination unit 16 described with reference to FIG. 3A is replaced in the apparatus of FIG. 3B by a pulsewave parameters and height determination unit 17 that converts the signal or code provided by height detecting unit 33 into a height measured from a reference point. In some embodiments, the reference point is selected using input from a user via operational input unit 22. For example, the reference point may be heart level, or it may be floor level (e.g., when the height is the cuff height, described with reference to FIGS. 1A and 2A) providing that the heart level does not change during the measurements. If the heart level does change during the measurements, the reference point is typically the heart level.

It is noted that in some embodiments, arterial parameters calculating unit 34 calculates arterial parameters of the subject without microprocessor 14 receiving any data regarding the height of the measuring site, i.e., without receiving data from height-related input unit 32, or height-detecting unit 33. For example, ASI and/or the PP-nonelastic/PP-elastic ratio calculated from ASI, using Eq. 13, may be calculated without microprocessor 14 receiving any data regarding the height of the pulsewave measuring site. In some embodiments, the placement of the measuring site at different heights by the user, serves as a tool for creating variability in BP. Therefore, it is not necessarily important for microprocessor 14 to receive data regarding the height of the measuring site.

Reference is now made to FIG. 4, which is a flowchart showing the operation of the blood pressure measurement apparatus, in accordance with an embodiment of the present invention. In some embodiments, after turning on the apparatus via operational input unit 22, an initiation process takes place (step ST1), during which the buffers involved in the measurements and calculations in pulsewave parameters determination unit 16, are cleared and the index n for the posture number receives the value 1 (step ST2). Subsequently, display unit 20 and/or height-related instructions generating unit 36 instruct the user to assume a posture following which, the user generates a START signal (step ST3). (Typically, operational input unit 22 comprises a START button which the user presses when ready.) In response to the START button being pressed, the apparatus activates pulsewave detection unit 10, and its output is digitized by A/D converter 12 and received by the pulsewave parameters determination unit 16 (step ST4). The determination unit calculates the pulsewave parameters (step ST5). These parameters may include systolic blood pressure (S) diastolic blood pressure (D), systolic and diastolic pulsewave velocity (typically calculated by measuring volume or pressure waveforms simultaneously at different locations), pulsewave pattern geometrical characteristics, as well as pulse volume (ΔV), pulse area, and/or pulse diameter. In step ST6 the resulting parameters are tested for acceptability, e.g., a test of acceptability may be that S or D should fall within a pre-determined range. A deviant value may be caused, for example, by organ movement during the measurement or improper cuff positioning. Typically, measurements can be deleted manually via the operational input unit 22, in case the user or the operator is aware of a problem and would like to repeat the measurement. In response, the measurement is deleted from data storage 18, and steps ST8 and ST9 result in instructions to repeat the deleted measurement. If parameters are found to be unacceptable, the apparatus returns to step (ST3).

For some applications, in step ST7 acceptable pulsewave parameters are stored in data storage 18 together with height-related data provided by height-related input unit 32, or height-detecting unit 33. Using the previously stored pulsewave parameters (if any), in step ST8, the apparatus determines if more measurements are desired for the determination of the physiological parameters using the parameters and their statistical significance calculated in step ST10 (more details about the process of calculating statistical significance are provided below). If more measurements are desired in order to calculate a physiological parameter, a new value m is applied to posture number n (step ST9) and the process returns to step ST3, in which display 20 displays a new posture (number m) and/or signals to the user to assume this posture and the display instructs the user to start the measurement.

Typically, the user is instructed to assume postures in a predetermined sequence e.g., postures 1 to 7 of FIG. 1A. In some embodiments, the user can override this automatic process by a manual selection of a posture via the operational input unit 22. In addition, in step ST10 the apparatus may identify a deviant measurement (which is not necessarily the previous measurement). For example, the apparatus may identify the deviant measurement by identifying that one of the pulsewave parameter readings deviates from a relationship established by the other pulsewave parameter readings. Typically, in response to identifying the deviant measurement, a signal is generated, instructing the user to repeat one or more measurements at preferred postures. In some embodiments, the arterial parameters calculating unit determines an arterial property of the subject without instructing the subject to repeat a measurement in response to determining the deviant measurement, for example, by not using the deviant measurement for determining the arterial property. Typically, when a set of physiological parameters are determined with sufficient accuracy (the set of parameters being as predetermined by the manufacturer or as pre-selected by the user and/or a healthcare professional via the operational input unit 22), the results of the calculations are displayed on display unit 20 and are automatically stored in the data storage 18 (step ST11).

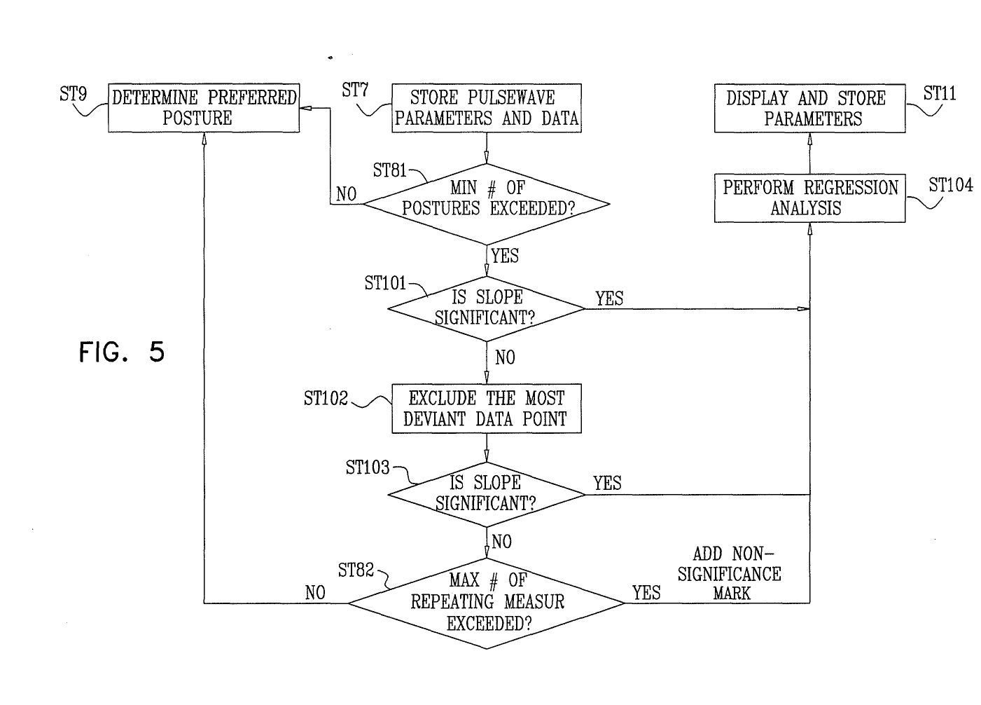

Reference is now made to FIG. 5, which is a flowchart showing the process of determining physiological parameters of a subject, in accordance with an embodiment of the present invention. Typically, physiological parameters are determined by linear regression of a Y versus X plot, in accordance with Eqs. 2, 4, 5, and 8. In some embodiments, the statistical significance of a slope is determined, in order to determine the statistical significance of a calculated physiological parameter.

The following statistical background may be helpful for understanding the calculation process: given n data points [X(i);Y(i)] (i=1, 2 . . . , n) hypothesized to fit a linear relationship with non-zero slope, a standard statistical method to test this hypothesis is to determine the correlation coefficient r defined as follows:

r²=σ²_{XY}=/(σ_{X}σ_{Y}) (Eq. 15)

where, σ²_{X}=<(X(i)−<X(i)>)²>, in which the brackets stand for the averaging operation i.e., summing over n terms and dividing the result by n.

Similarly,

σ²_{Y}=<(Y(i)−<Y(i)>)²>, and

σ²_{xY}=<(Y(i)−<Y(i)>)(X(i)−<X(i)>)>

σ_{X} and σ_{Y} are the standard deviations of the X and Y data respectively. The value of r ranges between 1 (a perfect correlation) to 0 (no correlation). The r value calculated for n data points relates to the significance p of the slope in the following way (see Sokal R R and Rohlf F J (1981) “Biometry” 2nd ed. Chap 15 pp. 561-616, Freeman, New York, which are incorporated by reference):

The parameter r²(n−2)/(1−r²) equals t², where t (taken from the well known t test) is a function of the significance level p, and n, and is found in standard statistical tables. If we start from given n and require p<0.05, one can determine r-critical values by expressing r² as a function oft as follows:

r²−critical=1/[((n−2)/t²)+1]

For r>r-critical, the slope is significant within p<0.05 level. The following table lists relevant data:

| | t (for | | |
|---|---|---|---|
| n | p = 0.05) | r²⁻critical | r-critical |
| 4 | 3.18 | 0.771 | 0.878 |
| 5 | 2.77 | 0.657 | 0.811 |
| 6 | 2.57 | 0.569 | 0.754 |
| 7 | 2.45 | 0.500 | 0.707 |
| 8 | 2.37 | 0.445 | 0.667 |
| 9 | 2.31 | 0.400 | 0.633 |

Alternatively, a similar method can be applied to nonlinear models, for example, to y=a+bx+cx², where a, b and c are determined by standard nonlinear regression methods that also provide a correlation coefficient r. In the case of nonlinear regression as well, r=1 corresponds to a perfect matching and r=0 to complete mismatching between the proposed model and the data. One can apply the same approach of pre-determining r-critical values to a non-linear model, as described hereinabove with respect to a linear model.

In some embodiments, the apparatus instructs the user to perform a sequence of measurements while the user assumes a number of postures (step ST3, shown in FIG. 4). The postures can follow a default pattern, the posture for each of the measurements being determined in step ST9, or can be chosen manually, by the user assuming a specific posture. Typically, the manual operation overrides the default sequence of preferred postures. The process of consecutive measurements ends when measurements done involve a predetermined minimum number of postures (step ST81). For some applications, the user can voluntarily perform a number of measurements at the same postures (selected manually), but the calculations are not performed unless enough different postures are involved in these measurements. In some embodiments, taking measurements at a number of different postures is utilized in order to measure a wide enough range of height-dependent pulsewave parameters. For some applications, the calculated value of r is compared to the corresponding stored r-critical value (step ST101). If r>r-critical, the apparatus performs regression analysis (step ST104) and the results are displayed and stored (step ST11). The same procedure may be applied to nonlinear regression models.

In some embodiments, linear regression analysis is performed in accordance with techniques described in an article by von Eye A, and Schuster C, entitled “Regression Analysis for Social Science,” Academic Press, San Diego, 1998, Chap 12, pp. 209-236 (“von Eye 1998”) and as described in Gavish 2008, cited hereinabove. Both of these articles are incorporated by reference. The slope of a linear regression line that models the relationship between S and D can be estimated by the slope derived by a standard regression divided by r, based on the findings described in Gavish 2008 that the slope calculated by a symmetric regression can be estimated by the slope derived by a standard regression divided by r. Since it is known in the art that the slope derived by a standard regression is expressed by r(σY/σX) it follows that the slope calculated by a symmetric regression can be estimated to be (σY/σX), in accordance with the findings of Gavish (2008).

The scope of the present invention includes using a measuring device to measure a first variable and a second variable and determining a linear relationship between the first variable and the second variable by dividing (a) a standard deviation of the first variable by (b) a standard deviation of the second variable. This determining step is typically carried out using a control unit. In some embodiments, the first and second variables are, respectively, the systolic and the diastolic blood pressure of a subject.

In some embodiments, alternative or additional methods of detecting a deviant point are used, using techniques known in the art. For example, the deviation of a point from a regression line may be determined using techniques described in U.S. Pat. No. 6,662,032 to Gavish, and/or using techniques described in von Eye 1998.

Typically, if it is determined that the slope is not significant, i.e., r≦r-critical for which p 0.05, the apparatus identifies and excludes the most deviant data point (step ST102). In some embodiments, this is done by eliminating the [X(j);Y(j)] data point and calculating r(j) using the remaining n−1 points (i=1 to n, but i< >j). The largest r(j) value is obtained upon excluding the most deviant data point. When r(j) is found to be greater than r-critical, corresponding to n−1 data points (step 103) regression analysis (step ST104) is applied as described before. In some embodiments, if r does not reach its critical value, the user is instructed to repeat the measurement found to be most deviant, in the appropriate posture (step ST9). Alternatively, the user is instructed to take an additional measurement in a posture of the user's choice. The deviant data point is replaced by a new one and the analysis is repeated, as long as the slope does not reach significance, until the number of repetitions reaches a predetermined maximum (step ST82). In some embodiments, when the number of repetitions reaches the maximum, the slope is analyzed and the result is displayed with a special mark for non-significance. In some embodiments, the user can voluntarily add measurements at a posture of the user's choice even after the number of repetitions has reached the maximum. Physiological parameters of the subject may be calculated in response to the voluntary measurements, providing that the results, which include the results of the voluntary measurements, are of statistical significance. Typically, for each of the parameters determined by the regression analysis it is possible to estimate the error of determination (for example using methods described in the von Eye 1998 article and in the Gavish 2008 article). In some embodiments, this error is stored and/or displayed.

In some embodiments, ST104 comprises estimating the blood pressure at the heart level posture using the regression parameters and the identification of the measurement done at the heart level. In some embodiments, determining the blood pressure at heart level in this manner is more accurate than standard averaging of a number of measurements, as a number of measurements done at different heights is involved, and the resulting regression line represents an averaging. For some applications, other pressure-dependent parameters, such as systolic arterial stiffness and/or diastolic arterial stiffness are determined at a heart level.

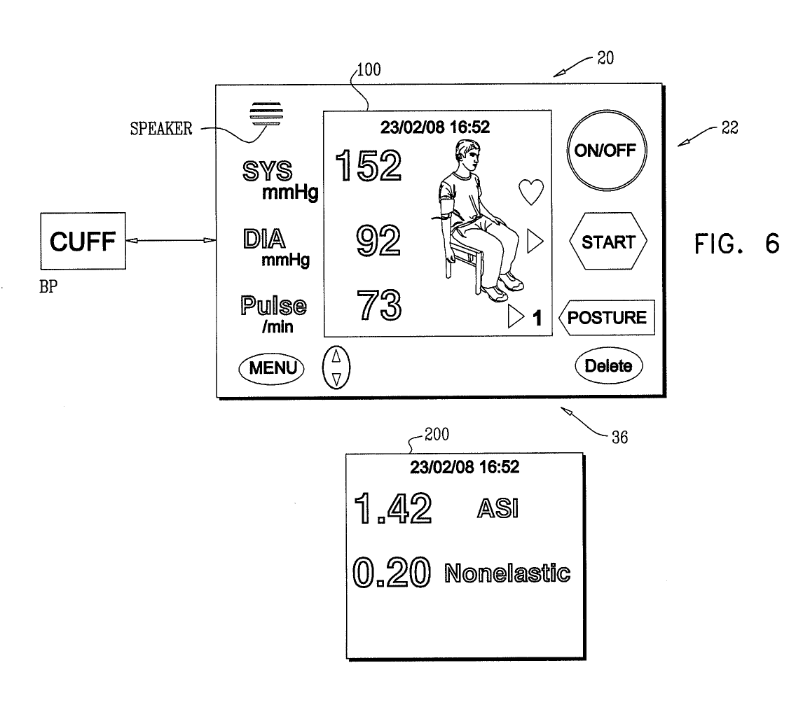

Reference is now made to FIG. 6, which is a schematic illustration of operational input unit 22, display unit 20, and height-related instructions generating unit 36, for use with a cuff that measures blood pressure, in accordance with an embodiment of the present invention. Typically, height-related instructions generating unit 36 instructs the user to assume different postures, and when the user has assumed the posture, pulsewave detection unit 10 (shown in FIG. 3A) measures blood pressure. In some embodiments, the apparatus includes a speaker to provide the user with voice instructions. Typically, the unit comprises one or more screens, as well as buttons for inputting data. For some applications, display unit 20 includes two types of screens: one type is used during the measurements (screen 100) and the other for reporting physiological parameters (screen 200). Typically, screen 100 displays the postures to be assumed by the user, e.g., the seven postures depicted in FIG. 1A, and screen 200 displays the name and units of the various variables displayed. This basic display structure is shared by all the embodiments shown.

In some embodiments, when an ON button of operational input unit 22 is pressed, measurement screen 100 is displayed. For some applications, the screen shows:

i) the posture selected as a default, numbered as “1,”

ii) a “posture marker” pointing towards this number, and/or

iii) the date and time at the top of the display.

In some embodiments, a speaker gives a voice instruction that describes a posture that the user should assume, e.g., “hang your hand down freely and when ready press START.” In some embodiments, during the voice instruction, the “posture marker” disappears and a “start marker” above blinks until the START button is pressed. In some embodiments, there is a lag between when instructions are given and when measurements are taken, in order to assure adequate restoration of the circulation.

In some embodiments, the user is instructed to assume a posture in which the blood pressure measuring site is at a heart level first. For some applications, for this specific posture a heart-like icon is displayed too. In some embodiments, upon the user pressing START, the apparatus starts the measurement and displays the values systolic BP, diastolic BP and pulse rate, where the corresponding labels “SYS” “DIA” and “Pulse” with suitable units are printed on the box cover in parallel locations. These parameters are typically stored together with the date and time and the posture number, unless the user presses the Delete button which erases the measurement result. In some embodiments, if an erroneous measurement is taken, an appropriate error message is displayed, e.g. ERR at the location of the systolic BP data. In some embodiments, a voice message provides a “corrective instruction,” e.g., “please do not move your hand during measurement,” or “please repeat the measurement.” In some embodiments, the first measurement is always repeated.

In some embodiments, a series of measurements at different postures is started by pressing POSTURE. Upon completing each measurement, the user presses the POSTURE button and the next posture is displayed. Pressing START before pressing POSTURE would repeat the measurement and add a new data point to the same posture. The process is typically repeated until measurements are performed in all designated postures. In some embodiments, pressing Delete after pressing POSTURE results in the current posture being ignored and a measurement being taken at the previous posture. This may be important when some postures are difficult to reach, e.g., hand raising, for people with limited hand movements, or when BP is too high, which may result in pain during measurement at the lowest sensor positions, or when the BP is too low, which results in failure of the device to measure at the highest sensor positions. During this process, the apparatus performs the data processing described with reference to FIG. 4 and FIG. 5.

This process results, for example, in the determination of the parameter ASI and/or the PP-nonelastic/PP-elastic ratio calculated from ASI using Eq. 13, which is displayed by the analysis screen 200 adjacent to where the screen says “nonelastic.” Typically, the different postures assumed by the user serve as a tool for creating variability in BP. Therefore, it is not necessarily important to assume the exact posture as displayed.

In some embodiments, the blood pressure at the heart level is calculated using the regression model, and displayed on the screen. For some applications, the average pulse rate is also displayed.

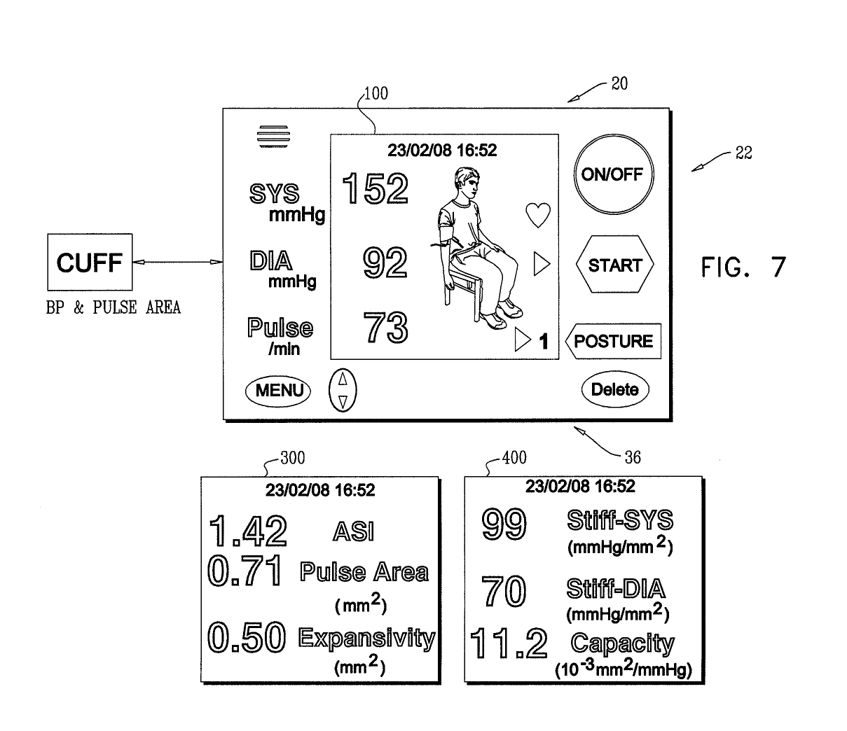

Reference is now made to FIG. 7, which is a schematic illustration of operational input unit 22, display unit 20, and height-related instructions generating unit 36, for use with a cuff that measures blood pressure and pulse volume, in accordance with an embodiment of the present invention. The unit typically includes analysis screens 300 and 400. (In some embodiments, data shown in screens 300 and 400 are all shown in a single screen.)

Analysis screens 300 and 400 differ from analysis screen 200 of FIG. 6, in that screens 300 and 400 additionally display additional pulsewave parameters, and/or additional arterial properties (in addition to ASI and/or the PP-nonelastic/PP-elastic ratio) that can be calculated from the additional pulsewave parameters that are measured. For example, the additional pulsewave parameters may include pulse area, pulse diameter, pulse volume, and/or arterial capacitance. (It is noted that the word “Capacity” that appears on screen 400, as shown in FIG. 7, represents arterial capacitance.) The additional arterial properties may include arterial expansivity, systolic arterial stiffness, diastolic arterial stiffness, and/or the zero-stiffness pressure. In some embodiments, some or all of the arterial properties are estimated at the heart level and displayed adjacent to a symbol representing heart level. In some embodiments, not all derived pulsewave parameters and/or arterial properties are displayed.

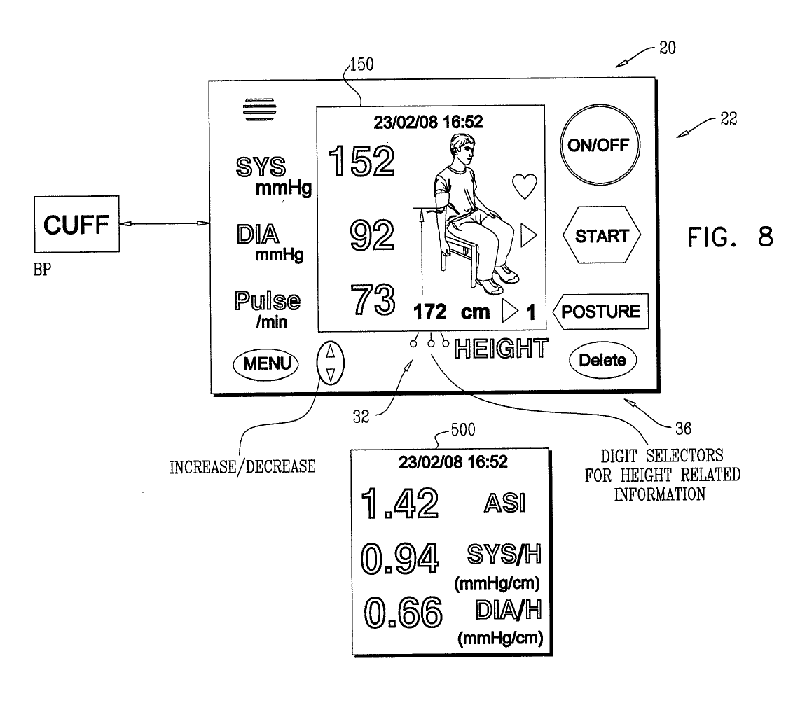

Reference is now made to FIG. 8, which is a schematic illustration of operational input unit 22, display unit 20, and height-related instructions generating unit 36, for use with a pulsewave detection unit 10 that measures blood pressure, and with a height-related input unit 32 for manually receiving information regarding the height of the blood pressure measuring site, in accordance with an embodiment of the present invention. The apparatus of FIG. 8 is generally similar to that of FIG. 6. The apparatus of FIG. 8 includes digit selectors for keying in a height indication, for example, in one of two ways: i) height is measured by the user and keyed in, or ii) codes that correspond to a height of a support structure, as described hereinbelow, are keyed in. The apparatus includes an analysis screen 500 which displays (in addition to ASI), for example, the derivative of systolic blood pressure with respect to height and the derivative of diastolic blood pressure with respect to height.

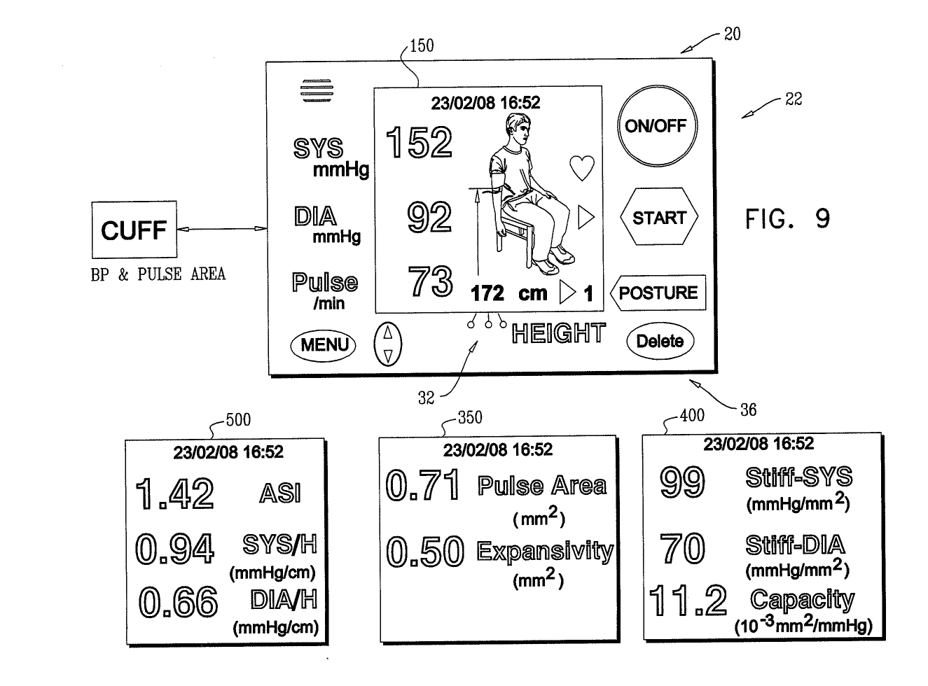

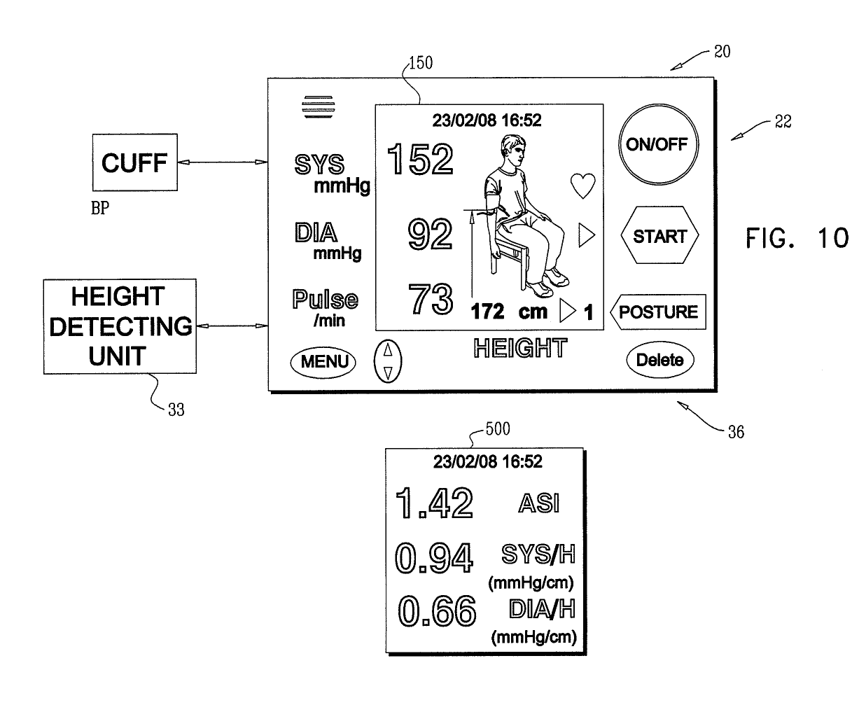

Reference is now made to FIG. 9, which is a schematic illustration of operational input unit 22, display unit 20, and height-related instructions generating unit 36, for use with a pulsewave detection unit 10 that measures blood pressure and pulse volume, and with a height-related input unit 32 for manually receiving information regarding the height of the blood pressure measuring site, in accordance with an embodiment of the present invention. In some embodiments, the apparatus includes analysis screens 350, 400, and 500, for displaying parameters which can be calculated using measurements of blood pressure and pulse volume at known heights.

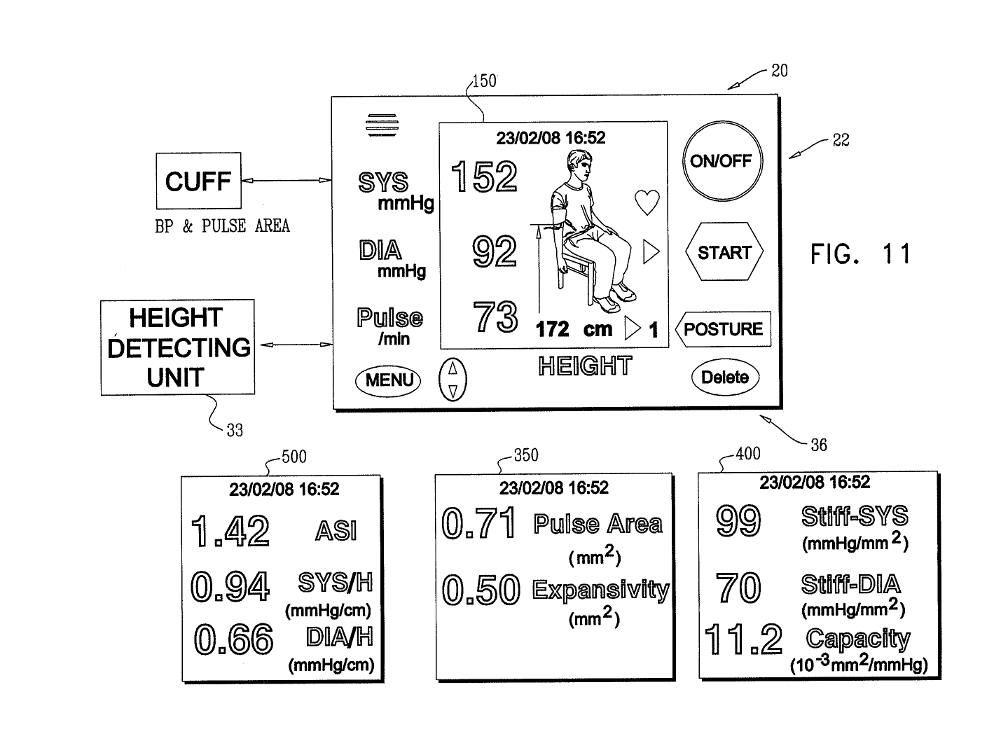

Reference is now made to FIG. 10, which is a schematic illustration of operational input unit 22, display unit 20, and height-related instructions generating unit 36, for use with a pulsewave detection unit 10 (shown in FIG. 3A) that measures blood pressure and with a height-detecting unit 33 for receiving information regarding the height of the blood pressure measuring site, in accordance with an embodiment of the present invention. The apparatus is generally similar to that described with respect to FIG. 8 with the following differences: i) the height of the pulsewave sensor is measured directly by the height-detecting unit, and ii) there are no keys for keying in height.

Reference is now made to FIG. 11, which is a schematic illustration of operational input unit 22, display unit 20, and height-related instructions generating unit 36, for use with a pulsewave detection unit 10 (shown in FIG. 3A) that measures blood pressure and pulse volume and with a height-detecting unit 33 for receiving information regarding the height of the blood pressure measuring site via a sensor, in accordance with an embodiment of the present invention. In some embodiments, the apparatus includes analysis screens 350, 400, and 500, for displaying parameters which can be calculated using measurements of blood pressure and pulse volume at known heights.

Reference is now made to FIG. 12, which is a schematic illustration of a support structure 40 for supporting a blood pressure measuring site, in accordance with an embodiment of the present invention. The support structure shown is designed for supporting a forearm with a wrist cuff for measuring blood pressure at different heights. Typically, the forearm support structure 40 includes a supporting arm 50 attached to a height-fixing rod 60, the height-fixing rod being kept at a vertical position and fixed in height by being attached to a base (not shown) or by being fixed firmly to a wall or any other stable structure (not shown). In some embodiments, the forearm supporter comprises two supporting arches 51 that are fixed by a fork-like holder 52 at distance that enables the user to place his/her forearm thereon.

Typically, an extension 53 of the fork-like holder 52 is inserted into a holder 54, in a way that it is free to rotate with variable protrusion (i.e., “telescopic” capability), as shown by arrows 57 and 59. The form of the supporting arches 51 is typically selected in a way that extension 53 points approximately to the center of gravity of the cuff. The holder 54 is fixed to the height-fixing rod 60 by a coupler that includes a position locker 56 that fixes the height of forearm supporter 40 by being pushed into one of the grooves 62 made at pre-determined heights on the rod 64, typically in 2-10 cm intervals (e.g., 5 cm intervals). The holder 54 with the coupler and the position locker 56 are free to rotate in the plane perpendicular to the height-fixing rod 60. As a result, the forearm supporter 40 provides the operator a convenient way to select a height H but leaves to the user all degrees of freedom required for finding a comfortable posture for placing the forearm at the selected height. The grooves 62 are marked by height-related codes. When the user is seated it is recommended that the heart level be close to the height of one of the grooves 62. In some embodiments, in case of a slight difference, between the heart level and the center of gravity of the cuff, the operator can place a thin pillow of height of ˜2.5 cm to reduce the difference.

In this way, the height-related code of the heart level generates a reference for measuring the pulsewave parameters at different predetermined heights characterized by the height-related codes. For example, if the height-related codes are numbered #1, #2, #3 . . . (as shown), where a unit change corresponds to a 5 cm height interval and heart level relates to code number 5, then placing the forearm in a position corresponding to code number 10 means that the cuff is 25 cm above the heart level.

The use of such an accessory is not limited to a wrist-type cuff. The principle of keeping many or all possible degrees of freedom for placing a limb with a cuff at a comfortable posture, while keeping the center of gravity of the cuff at a predetermined height, can be implemented in many other ways. Since different people may differ considerably in the thickness of the arm or the wrist, there may be a number of supporting arms 50 that differ by the depth of the supporting arches 51 with respect to the height of the holder 54, or a single model of supporting arm 50 may be provided with an appropriate arrangement for adjusting this variable (not shown).

Although the height-related codes (or the heights themselves) can be keyed in, as described with respect to FIG. 8 and FIG. 9, in some embodiments, the code is transmitted to the apparatus electronically. One embodiment is shown on the right-hand side of the figure: the rod 64 of the forearm supporter 40 includes a series of resistors (R) in a way that the connection of the supporting arm 50 to the rod 60 generates resistance that increases linearly with the height-related code. This resistance serves as an input to the apparatus, which converts the resistance into the corresponding height. In that way, the forearm supporter 40 acts as the sensing component of the height-detecting unit 33, and the apparatus interface is as illustrated in FIG. 10 or FIG. 11. In some embodiments, a height-detecting unit, as described herein, is coupled to forearm supporter 40, and detects the height of the forearm supporter, in accordance with the techniques described herein.

Although embodiments have been described in which a pulsewave detection unit detects pulse volume, the scope of the present invention includes a pulsewave detection unit that detects other pulsewave parameters that are directly related to pulse volume, for example, pulse area and pulse diameter.

Although embodiments have been described in which a height indication is detected, or is input to a height-related input unit, in some embodiments, an actual height is detected, and/or input to the height-related input unit, for example, using a position sensor, an acceleration sensor, an ultrasound detector, and/or using a different method. In some embodiments, one or more of the aforementioned sensors is coupled to pulsewave detection unit and measures the height of at least a portion of the pulsewave detection unit that is coupled to a portion of the subject's body. For example, a sensor may be coupled to a blood pressure cuff that is coupled to a subject's arm.

It is also to be understood that although some embodiments of the present invention described hereinabove utilize height as an input (in one form or another), the scope of the present invention includes the determination of blood vessel characteristics in the absence of a height input, and, for example, determining blood vessel characteristics based on multiple measurements recorded at different heights, without indicating the specific heights at which the measurements were taken.

It is also to be understood that although some embodiments of the present invention described hereinabove, and claimed hereinbelow, describe a blood pressure sensor, the scope of the term “blood pressure sensor” includes any sensor that generates a signal responsively to arterial pressure, for example, a blood pressure measuring cuff, a photoplethysmograph, and/or any other sensor for generating an indication responsively to arterial pressure that is known in the art.

It is noted that for some applications, the methods described hereinabove may be performed using software (e.g., software provided on a computer-readable disk, or on another computer-readable medium) that is used in conjunction with apparatus that is known in the art. For example, software may be provided that performs some or all of the steps provided in the flowcharts shown in FIGS. 4 and 5, when used in conjunction with a standard computer and a standard blood pressure monitor (or another standard pulsewave detection unit).

For example, a standard blood pressure monitor (and/or another standard pulsewave detection unit) may be used to perform functionalities of one or more of the following components that are described hereinabove: pulsewave detection unit 10, pulsewave parameters determination unit 16, height-related input unit 32, and/or height-related detection unit 33. Dedicated software may be provided to facilitate the performance of the functionalities of arterial-parameters calculating unit 34 by the microprocessor of a standard computer that is in communication with the blood-pressure monitor. For some applications, the software facilitates the performance of functionalities of height-related instructions unit 36 by the computer. Typically, a monitor that is in communication with the computer performs functionalities of display unit 20.

In general, it will be understood by one skilled in the art that aspects of the present invention described hereinabove can be embodied in a computer running software, and that the software can be supplied and stored in tangible media, e.g., on a disk, or in intangible media, e.g., in an electronic memory, or on a network such as the Internet.

It will be appreciated by persons skilled in the art that the present invention is not limited to what has been particularly shown and described hereinabove. Rather, the scope of the present invention includes both combinations and sub combinations of the various features described hereinabove, as well as variations and modifications thereof that are not in the prior art, which would occur to persons skilled in the art upon reading the foregoing description.

1. Apparatus, comprising:
a pulsewave detection unit, at least a portion of which is configured to be coupled to a portion of a subject's body, the pulsewave detection unit being configured to generate a signal that is responsive to arterial pressure of the portion of the subject's body; and
a control unit comprising:
a pulsewave parameters determination unit configured to receive respective first and second signals from the pulsewave detection unit, the signals being responsive to arterial pressure of the portion of the subject's body while the portion of the pulsewave detection unit that is coupled to the portion of the subject's body is at respective first and second heights, at respective times; and
an arterial parameters calculating unit configured to determine an arterial property of the subject by processing the first and second signals, and to generate an output in response to determining the arterial property.

2-4. (canceled)

5. The apparatus according to claim 1, wherein the pulsewave detection unit comprises a sensor selected from the group consisting of a photoplethysmographic sensor, a strain gauge plethysmograph, a cuff configured to be placed on the portion of the subject's body and to generate the signal responsively to changes in blood pressure of the subject, an intravascular pressure sensor, and a sensor configured to generate the signal responsively to measuring a spectral property of blood of the subject.

6-8. (canceled)

9. The apparatus according to claim 1, wherein the arterial parameters calculating unit is configured to facilitate the generation of the first and second signals while the portion of the pulsewave detection unit is at respective first and second heights by instructing the subject to move the portion of the subject's body to which the portion of the pulsewave detection unit is coupled, to respective first and second heights.

10-13. (canceled)

14. The apparatus according to claim 1, further comprising a height-related input unit coupled to the control unit, wherein the control unit is configured to receive height indications regarding the first and second heights from the subject via the height-related input unit, and the arterial parameters calculating unit is configured to determine the arterial property of the subject by processing the first and second signals and the height indications regarding the first and second heights.

15. The apparatus according to claim 1, further comprising a height-detecting unit configured to detect the first and second heights, wherein the control unit is configured to receive the height indications regarding the first and second heights from the height-detecting unit, and the arterial parameters calculating unit is configured to determine the arterial property of the subject by processing the first and second signals and the height indications regarding the first and second height.

16. The apparatus according to claim 15, wherein the height-detecting unit comprises a sensor selected from the group consisting of:
a position sensor coupled to the portion of the pulsewave detection unit that is coupled to the portion of the subject's body, and configured to generate a signal indicative of a spatial position of the portion of the pulsewave detection unit,
an acceleration sensor coupled to the portion of the pulsewave detection unit that is coupled to the portion of the subject's body, and configured to generate a signal indicative of a spatial position of the portion of the pulsewave detection unit,
an ultrasound detector coupled to the portion of the pulsewave detection unit that is coupled to the portion of the subject's body, and configured to generate a signal indicative of a spatial position of the portion of the pulsewave detection unit, and
a support structure configured to support the portion of the subject's body to which the portion of the pulsewave detection unit is coupled during the receiving of the signals, wherein the height-detecting unit is configured to measure a height of a portion of the support structure, the height of the portion of the support structure being related to the height of the portion of the pulsewave detection unit.

17-25. (canceled)

26. The apparatus according to claim 1,
wherein the pulsewave parameters determination unit is further configured to receive, from the pulsewave detection unit, at least third and fourth signals that are responsive to arterial pressure of the portion of the subject's body, while the portion of the pulsewave detection unit that is coupled to the portion of the subject's body is at respective third and fourth heights with respect to the subject's heart, and
wherein the arterial parameters calculating unit is configured:
to determine, based upon the received signals, first, second, third, and fourth pulsewave parameters of the subject, the parameters corresponding to the respective heights,
to identify that one of the pulsewave parameters deviates from a relationship established by the other pulsewave parameters, and
not to use the pulsewave parameter that deviates from the relationship, in determining the arterial property of the subject.

27. The apparatus according to claim 26, wherein the arterial parameters calculating unit is configured to generate an output signal to the subject indicating that the subject should repeat a measurement, in response to the arterial parameters calculating unit determining that one of the pulsewave parameters deviates from the relationship.

28. (canceled)

29. The apparatus according to claim 1, wherein the arterial parameters calculating unit is configured to determine the arterial property by determining a linear relationship between a first variable and a second variable by regression analysis, the first and second variable being based upon the first and second signals.

30. (canceled)

31. The apparatus according to claim 29, wherein the first variable includes a variable selected from the group consisting of systolic blood pressure, and diastolic blood pressure, and the second variable includes a variable selected from the group consisting of diastolic blood pressure, and height and wherein the arterial parameters calculating unit is configured to determine the linear relationship by determining a linear relationship between the selected first variable and the selected second variable.

32. The apparatus according to claim 31, wherein the arterial parameters calculating unit is configured to determine an arterial stiffening index of the subject based upon the determined linear relationship.

33. The apparatus according to claim 31, wherein the arterial parameters calculating unit is configured to determine blood pressure of the subject at a heart-level of the subject based on the linear relationship.

34. (canceled)

35. The apparatus according to claim 29, wherein the linear relationship that is determined by the regression analysis includes a slope between the first variable and the second variable, and wherein the arterial parameters calculating unit is configured to determine the slope between the first variable and the second variable by dividing the standard deviation of the first variable, by the standard deviation of the second variable.

36. (canceled)

37. The apparatus according to claim 29, wherein the arterial parameters calculating unit is configured to identify a deviant data point by:
calculating a first correlation coefficient of the linear relationship between the first variable and the second variable,
removing a data point and calculating a second correlation coefficient of the linear relationship between the first variable and the second variable, the data point having been removed, and
comparing the first correlation coefficient to the second correlation coefficient.

38-48. (canceled)

49. The apparatus according to claim 1,
wherein the pulsewave detection unit comprises a pulse sensor configured to generate a pulse volume signal that is related to a pulse volume of the subject,
wherein the pulsewave parameters determination unit is configured to receive respective first and second pulse volume sensor signals from the pulse volume sensor while the portion of the pulsewave detection unit that is coupled to the portion of the subject's body is at the respective first and second heights with respect to the heart of the subject, and
wherein the arterial parameters calculating unit is configured to determine the arterial property of the subject by processing the first and second blood pressure sensor signals and the first and second pulse volume signals.

50-53. (canceled)

54. A method, comprising:
receiving a first signal that is responsive to arterial pressure of a subject at a first time, while at least a portion of a device that generates the signal, while coupled to a portion of a body of the subject, is at a first height with respect to a heart of the subject;
receiving a second signal that is responsive to arterial pressure of the subject at a second time, while the portion of the device is at a second height with respect to the subject's heart;
determining an arterial property of the subject by processing the first and the second signals; and
generating an output in response to determining the arterial property.

55-56. (canceled)

57. The method according to claim 54, wherein receiving the first and the second signals comprises performing an action selected from the group consisting of:
receiving the signals responsively to photoplethysmographically measuring a blood volume of the subject,
receiving the signals from a strain gauge plethysmograph,
receiving the signals from a cuff placed on the portion of the subject's body, the cuff generating the signals responsively to changes in blood pressure of the subject,
receiving the signal responsively to measuring blood pressure of the subject intravascularly,
receiving the signal responsively to measuring a spectral property of blood of the subject.

58-59. (canceled)

60. The method according to claim 54, further comprising:
determining the first and the second height; and
generating a height-output in response to determining the heights.

61-65. (canceled)

66. The method according to claim 54,
further comprising:
receiving height indications regarding the first and second heights from a user,
wherein determining the arterial property of the subject comprises determining the arterial property by processing the first and second signals and the height indications regarding the first and second heights.

67. The method according to claim 54,
further comprising:
receiving height indications regarding the first and second heights from a height-detecting unit,
wherein determining the arterial property of the subject comprises determining the arterial property by processing the first and second signals and the height indications regarding the first and second heights.

68. The method according to claim 67, wherein the height-detecting unit includes a height-detecting unit selected from the group consisting of: a position sensor, an acceleration sensor, an ultrasound sensor, and a fluid-pressure sensor, and wherein receiving the indications from the height-detecting unit comprises receiving the indication from the selected height-detecting unit.

69-72. (canceled)

73. The method according to claim 54, further comprising:
receiving third and fourth signals that are responsive to arterial pressure of the subject, while the portion of the device, while coupled to the portion of the subject's body, is at respective third and fourth heights;
based upon the received signals, determining first, second, third, and fourth pulsewave parameters of the subject, the parameters corresponding to the respective heights;
identifying that a pulsewave parameter deviates from a relationship established by the other pulsewave parameters; and
not using the pulsewave parameter that deviates from the relationship in determining the arterial property of the subject.

74. The method according to claim 73, further comprising, in response to determining that one of the pulsewave parameters deviates from the relationship, generating an output signal to the subject indicating that the subject should repeat a measurement.

75. (canceled)

76. The method according to claim 54, wherein determining the arterial property comprises determining a linear relationship between a first variable and a second variable by regression analysis, the first and second variable being based upon the first and second signals.

77. (canceled)

78. The method according to claim 76, wherein the first variable includes a variable selected from the group consisting of systolic blood pressure, and diastolic blood pressure, and the second variable includes a variable selected from the group consisting of diastolic blood pressure, and height, and wherein determining the linear relationship comprises determining a linear relationship between the selected first variable and the selected second variable.

79. The method according to claim 78, wherein determining the arterial property comprises determining blood pressure of the subject at a heart-level of the subject.

80. The method according to claim 78, wherein determining the arterial property comprises determining an arterial stiffening index of the subject from the determined linear relationship.

81. The method according to claim 76, wherein determining the linear relationship between the first variable and the second variable comprises dividing a standard deviation of the first variable, by a standard deviation of the second variable.

82-83. (canceled)

84. The method according to claim 54, further comprising identifying a deviant data point by:
calculating a first correlation coefficient of the linear relationship between the first variable and the second variable;
removing a data point and subsequently calculating a second correlation coefficient of the linear relationship between the first variable and the second variable, the data point having been removed; and
comparing the first correlation coefficient to the second correlation coefficient.

85-95. (canceled)

96. The method according to claim 54, further comprising:
receiving a first and a second pulse signal that indicates pulse volume of the subject, while the portion of the device, while coupled to the portion of the subject's body, is respectively at the first and the second heights,
wherein determining the arterial property comprises determining the arterial property by processing the first and second blood pressure signals and the first and second pulse signals.

97-99. (canceled)

100. A computer program product for use with a pulsewave detection unit, at least a portion of which pulsewave detection unit is configured to be coupled to a portion of a subject's body, the pulsewave detection unit being configured to generate a signal that is responsive to arterial pressure of the portion of the subject's body, the product comprising a computer-readable medium having program instructions embodied therein, which instructions, when read by a computer, cause the computer to:
receive respective first and second signals from the pulsewave detection unit, the signals being generated responsively to arterial pressure of the subject at respective times, while the portion of the pulsewave detection unit that is coupled to the portion of the subject's body is at respective first and second heights,
determine an arterial property of the subject by processing the first and second signals, and
generate an output in response to determining the arterial property.
